(19) United States
(12) Patent Application Publication
(10) Pub. No.: US 2020/0253491 A1
(43) Pub. Date: Aug. 13, 2020

(54) Mobile Wearable Combinatorial Ultrasound/Near Infrared Sensor System

(71) Applicant: Brown University, Providence, RI (US)

(72) Inventor: Arto NURMIKKO, Providence, RI (US)

(21) Appl. No.: 16/789,208

(22) Filed: Feb. 12, 2020

Related U.S. Application Data
(60) Provisional application No. 62/804,455, filed on Feb. 12, 2019.

(51) Int. Cl. A61B 5/026; A61B 5/00; G16H 40/67; G16H 80/00

(52) U.S. Cl. CPC A61B 5/0261; A61B 5/6822; A61B 5/0024; A61B 5/0022; A61B 5/002; A61B 2562/0238; G16H 80/00; A61B 2562/0204; A61B 2562/0219; A61B 2562/0271; G16H 40/67

(57) ABSTRACT

An apparatus includes a mobile, neck wearable combinatorial ultrasound/near infrared sensors system configured for detection of embolic events in carotid arteries.

### CROSS REFERENCE TO RELATED APPLICATIONS

This application claims benefit from U.S. Provisional Patent Application Ser. No. 62/804,455, filed Feb. 12, 2019, which is incorporated by reference in its entirety.

### STATEMENT REGARDING GOVERNMENT INTEREST

None.

### BACKGROUND OF THE INVENTION

The present invention relates generally to sensors, and more particularly to a mobile wearable combinatorial ultrasound/near infrared sensor system.

In general, a major challenge in ischemic stroke care is the fact that treatments are reactive in their nature and must be initiated within hours following the onset. The only FDA approved pharmaceutical treatment of ischemic stroke in the form of recombinant tissue plasminogen activator (rTPA) administration must be initiated within 4.5 hrs post-stroke to be effective. However, only about 25% of stroke victims reach the hospital within 3 hours and the onset-to-door time has remained largely unchanged over the course of past decades.

### SUMMARY OF THE INVENTION

The following presents a simplified summary of the innovation in order to provide a basic understanding of some aspects of the invention. This summary is not an extensive overview of the invention. It is intended to neither identify key or critical elements of the invention nor delineate the scope of the invention. Its sole purpose is to present some concepts of the invention in a simplified form as a prelude to the more detailed description that is presented later.

In general, in one aspect, the invention features a system including a hemodynamic neck device configured to overlay on an emboli-carrying common carotid artery in a human neck, a vest electronics system linked to the hemodynamic neck device, andca wireless communication system linked to the vest electronics system.

In another aspect, the invention features an apparatus including a mobile, neck wearable combinatorial ultrasound/near infrared sensors system configured for detection of embolic events in carotid arteries.

The invention may include one or more of the following advantages.

The present invention is a user-friendly system which (a) deploys miniature arrays of US-NIRS transducers for transcutaneous detection of microembolic events in common carotid arteries and (b) enables their early classification by wearable custom signal processor—all as a mobile, wireless medical device system.

The present invention is applicable to a range of clinically relevant scenarios for people at finite risk of stroke, in particular during the early post-stroke time window associated with the high risk of reoccurrence.

These and other features and advantages will be apparent from a reading of the following detailed description and a review of the associated drawings. It is to be understood that both the foregoing general description and the following detailed description are explanatory only and are not restrictive of aspects as claimed.

### BRIEF DESCRIPTION OF THE DRAWINGS

These and other features, aspects, and advantages of the present invention will become better understood with reference to the following description, appended claims, and accompanying drawings where:

FIG. 1 is a block diagram of an exemplary hemodynamic neck device.

FIG. 2 is a block diagram of an exemplary microarray.

FIG. 3 is a block diagram of exemplary vest electronics.

FIG. 4 is a block diagram of an exemplary wireless system and a remote health system.

### DETAILED DESCRIPTION

The subject innovation is now described with reference to the drawings, wherein like reference numerals are used to refer to like elements throughout. In the following description, for purposes of explanation, numerous specific details are set forth in order to provide a thorough understanding of the present invention. It may be evident, however, that the present invention may be practiced without these specific details. In other instances, well-known structures and devices are shown in block diagram form in order to facilitate describing the present invention.

Doppler ultrasound detection of emboli in cerebral arteries using hand-held or head-mountable probes is in clinical use today. The transcranial Doppler-based detection of emboli has shown that more prolonged monitoring enables identification of the patients which on the short term would be considered as free of embolism. Due to the limitations of the transcranial Doppler technology, the longest monitoring reported to this date was 6 hours long, whereas the duration of the typical monitoring procedure is around 15 minutes and is restricted to the patients with optimal temporal bone window. Furthermore, the geometry of the middle cerebral arteries relative to the skull even though is perfect for the single transducer Doppler measurements, does not favor embolus interrogation with multiple transducers for accurate size and composition characterization. The present invention introduces a concept of patient-specific wearable US-NIRS technology suited for 24/7 unobtrusive monitoring of the carotid arteries in the neck. The device is tailored to particular patient's anatomy and physiology to allow continuous tracking of the target blood vessel with optimal signal-to-noise ratio, false-positive event rate and power consumption.

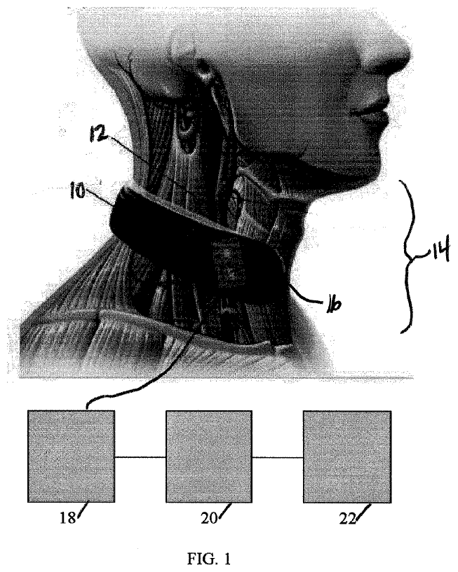

In FIG. 1, an exemplary hemodynamic neck device 10 is shown overlaid on an emboli-carrying common carotid artery 12 in a human neck 14. A microarray 16 as embedded in the hemodynamic neck device 10 and linked to a vest electronics system 18. The vest electronics system 18 includes or is linked to a wireless communication system 20 that can communicate with a remote health monitor 22, such as a smartwatch, and/or with a clinician or clinical site.

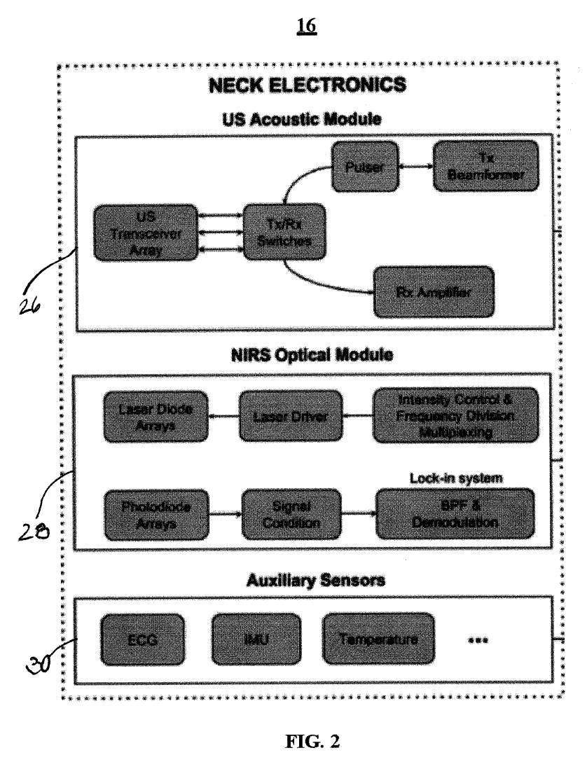

The microarray 16 in the hemodynamic neck device 10 includes a US-NIRS microarray patch configuration with “sentry” sensors operating continuously for event detection and “interrogator” sensors brought online to verify and characterize potential events. More specifically, as shown in FIG. 2, the microarray 16, also referred to as neck electronics, includes a US acoustic module 26, a NIRS optical module 28 and auxiliary sensors 30.

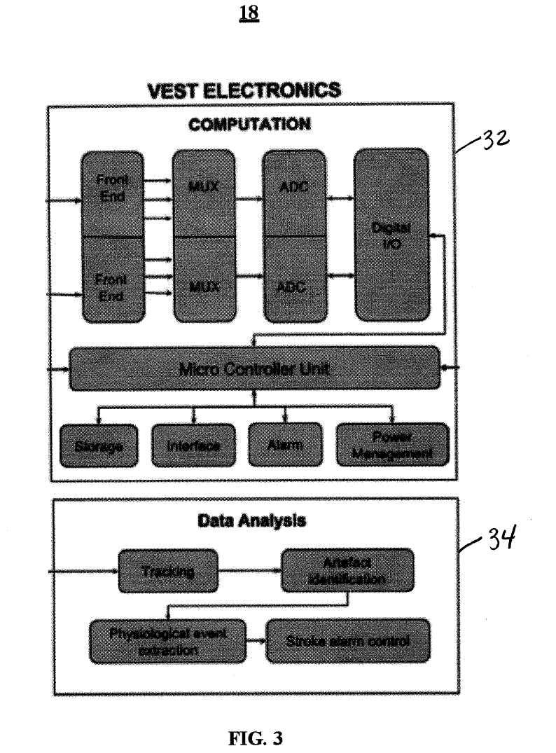

As shown in FIG. 3, the vest electronics system 18 includes computation module 32 and a data analysis module 34. The modules 32, 24 are configured to implement classifying algorithms and encrypted telecommunications to be relayed to the wireless communication system 20.

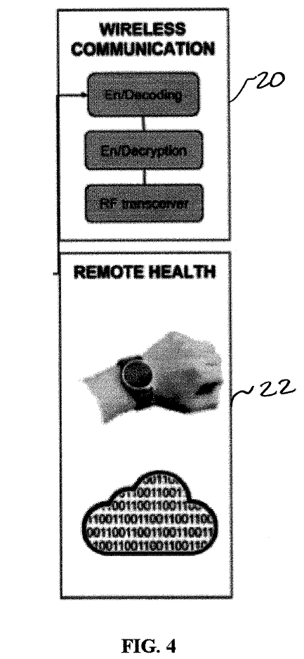

In FIG. 4, block diagrams of wireless communication system 20 and the remote health monitor 22 are illustrated.

Referring again to FIG. 1, the multiple components 10, 16, 18, 20, 22 as parts provide a single wearable device aimed at maximally comprehensive continuous interrogation of the patient's hemodynamic state. A key component is US-NIRS based, transcutaneous, vascular microemboli detector/classifier, configured for 24/7 wearable use. The linear arrays of miniaturized NIRS sensors/emitters and US transducers are lined up along the carotid arteries around the neck area, actively guided by the NIRS detection of blood pressure. An array of NIR lasers sends in the light to interrogate the hemodynamic state of the blood flow detected as the backscattered photons by the array of NIR sensors configured for monitoring across multiple depths and directions across the artery. The NIR measurements are coupled with US interrogation of the blood flow. A piezoelectric transducer emits sub-microsecond duration ultrasound pulses to the target blood vessel. The backscattered acoustic signals are detected by the piezoelement array in a time-domain mode of particle detection.

Besides using the system described above for stroke forecasting and risk stratification, the present invention holds the potential for providing critical insights into contemporary clinical research questions of ischemic stroke. First, even though transcranially detected emboli were found to correlate with early ischemic recurrence, and stroke risk in asymptomatic carotid stenosis patients, the full mechanism and true predictive power of microembolism remains to be an open question especially for the emboli of cardiac etiology. For instance, the long-term real-life dynamics of embolism and its correlation with a life-style and everyday activities is not known. Second, there has been very little progress in the attempts of extracting the emolic size information, and the typical clinical microembolic examination is still binary by its nature—the embolus is either present or not. There has been even less progress on composition characterization. Despite the fact that the diversity of embolic composition has been acknowledged, the main focus in this direction has been on differentiation between solid and gas emboli. We think that one of the reasons for previously reported relatively small increase in ischemic stroke risk in the presence of emboli is due to the reliance purely on embolic counts, ignoring the size and composition information. The present invention relies on interrogation of carotid arteries in the neck, which provides a perfect geometrical configuration for more detailed interrogation of the trespassing embolus by employing an array of US-NIRS task-specific sensors. Third, the fact that we envision the monitoring of other physiological quantities besides emboli (e.g., blood flow, ECG, body motion, oxygenation), opens an unprecedented possibility of glimpsing into the bodily activity in the midst or prior to stroke. For instance, the long-term knowledge about cardiac function and embolic patterns can facilitate the diagnosis of embolic etiology following the stroke and alleviate the need for transesophageal echocardiography, cardiac computed tomographic angiography or implantable cardiac monitors traditionally used for the detection of a cardiac source of embolism. Furthermore, the availability of such cardiovascular health big-data can allow identification of unique stroke signatures and patient classification. Such classification could then land itself into more personalized stroke management strategies. Fourth, the dynamics, prevalence and impact of the micro-strokes are very poorly understood. By monitoring the emboli in carotid arteries, it is possible to identify the emboli remaining within the brain without causing a major stroke and investigate the implications of such events to the patient's dynamic physiological function. Finally, the idea of immediate pharmaceutical intervention, even though far in the future (due to a large number of comorbidities which must be accounted for before making the decision on reactive treatment initiation) offers the avenue for developing a better understanding of physiological responses to more immediate chemical modulation, including interventions preceding the stroke onset.

In the present invention, a detection strategy of emboli and their identification (and possible classification), with on board electronic signal processing is based on a two-stage approach: (i) to first order, the combined UST/NIRS sensor array (in the neck patch) is a transesophageal particle counting device. The finite array (N approximately 10 elements, limited by combined anatomical and microdevice integration constraints) provides a statistically finite set of data for event verification in conjunction with real-time signal processing. (ii) to second order, the NIRS and UST sensors provide data for fingerprinting changes in local hemodynamic structures. Major arteries are complaint flow tubes so that e.g. NIRS signal will read out the arterial blood pressure, including the temporal variations according to the heart beat. Since the systolic-diastolic pressure change can be measured by conventional instruments (though somewhat time integrated), then the NIRS signal from the first “sentry” can provide a calibration and baseline of the signal measured by the subsequent US and NIRS sensors e.g. for background signal rejection. Tracking the blood pressure variations in the carotid artery thus provide quantitative scale while automatically taking account the compliance of the carotid artery for a given subject (stiffer artery—more plaque, etc). Then, when emboli transit the interrogation zone, not only is a particle detected and cross-correlated between the NIRS and US modalities but this calibration should give an estimate of the emboli size because of the local arterial pressure-induced variations induced by a particle whose diameter d is some fraction of the arterial diameter. That is, if the average diameter of the artery is D, we seek to measure the quantity ΔD (t,z)/D(z) which is a direct measure due to local fluid dynamical pressure changes induced on the compliant vessel walls by a (possibly small) transient “blockage” displacing local blood volume/flow. Our experiments suggest that given state-of-the art commercial opto- and ultrasonic devices, a measurable ΔD(t,z)/D(z) should be possible for ratios as small as d/D approximately 0.1.

In one embodiment, signal processing components are added to the system to correct for artifacts from movement, spatial slipping of the collar and its resetting back to the right target.

It would be appreciated by those skilled in the art that various changes and modifications can be made to the illustrated embodiments without departing from the spirit of the present invention. All such modifications and changes are intended to be within the scope of the present invention except as limited by the scope of the appended claims.

What is claimed is:

1. A system comprising:
a hemodynamic neck device configured to overlay on an emboli-carrying common carotid artery in a human neck;
a vest electronics system linked to the hemodynamic neck device; and
a wireless communication system linked to the vest electronics system.

2. The system of claim 1 wherein the hemodynamic neck device comprises a miniaturized combinatorial ultrasound/near infrared sensor system (NIRS).

3. The system of claim 2 wherein the miniaturized combinatorial ultrasound/near infrared sensor system comprises:
a US acoustic module;
a NIRS optical module; and
auxiliary sensors.

4. The system of claim 3 wherein the US acoustic module comprises:
a US Transceiver array communicatively linked to transmit/receive switches;
a pulsar linked to a transmit beamformer, the pulsar communicatively linked to transmit/receive switches; and
a receive amplifier communicatively linked to transmit/receive switches.

5. The system of claim 4 wherein the NIRS optical module comprises:
an intensity control and frequency division multiplexing module communicatively linked to a laser driver module;
a laser diode arrays module communicatively linked to laser driver module;
a BPF and demodulation module communicatively linked to a signal condition module; and
a photodiode arrays module communicatively linked to the signal condition module.

6. The system of claim 5 wherein the auxiliary sensors comprise:
an electrocardiogram sensor;
an inertial measurement unit sensor; and
a temperature sensor.

7. The system of claim 1 wherein the vest electronics system comprises:
a computation module; and
a data analysis module communicatively linked to a data analysis module.

8. The system of claim 7 wherein the data analysis module comprises:
a racking unit communicatively linked to an artefact identification unit;
a physiological event extraction unit communicatively linked to the artefact identification unit; and
a stroke alarm control unit communicatively linked to the physiological event extraction unit.

9. The system of claim 1 wherein the wireless communication system comprises:
an en/decoding unit;
an en/decryption unit; and
radio-frequency (RF) transceiver.

10. The system of claim 1 further comprising a remote health system wirelessly linked to the a wireless communication system.

11. The system of claim 10 wherein the remote health system is a smartwatch.

12. The system of claim 10 wherein the remote health system is a clinician's office.

13. The system of claim 10 wherein the remote health system is a hospital.

14. An apparatus comprising a mobile, neck wearable combinatorial ultrasound/near infrared sensors system configured for detection of embolic events in carotid arteries.

15. The system of claim 14 wherein the mobile, neck wearable combinatorial ultrasound/near infrared sensors system comprises:
a US acoustic module;
a NIRS optical module; and
auxiliary sensors.

16. The system of claim 15 wherein the US acoustic module comprises:
a US Transceiver array communicatively linked to transmit/receive switches;
a pulsar linked to a transmit beamformer, the pulsar communicatively linked to transmit/receive switches; and
a receive amplifier communicatively linked to transmit/receive switches.

17. The system of claim 16 wherein the NIRS optical module comprises:
an intensity control and frequency division multiplexing module communicatively linked to a laser driver module;
a laser diode arrays module communicatively linked to laser driver module;
a BPF and demodulation module communicatively linked to a signal condition module; and
a photodiode arrays module communicatively linked to the signal condition module.

18. The system of claim 5 wherein the auxiliary sensors comprise:
an electrocardiogram sensor;
an inertial measurement unit sensor; and
a temperature sensor.
